SEQUENCE LISTING (1 sequence)

SEQ ID NO: 1 - protein, 14 aa, synthetic construct
GKPIPNPLLGLDST

(19) United States
(12) Patent Application Publication
(10) Pub. No.: US 2026/0118350 A1
(43) Pub. Date: Apr. 30, 2026

(54) Fluorescent Probes for Peroxidase-Mediated Fluorescent Signal Amplification

(71) Applicant: Seoul National University R&DB Foundation, Seoul (KR)

(72) Inventor: Hyun Woo RHEE, Seoul (KR)

(73) Assignee: Seoul National University R&DB Foundation, Seoul (KR)

(21) Appl. No.: 19/033,778

(22) Filed: Jan. 22, 2025

(30) Foreign Application Priority Data
Jan. 22, 2024 (KR) 10-2024-0009606

(51) Int. Cl. G01N 33/533; G01N 33/68

(52) U.S. Cl. CPC G01N 33/533; G01N 33/6803

(57) ABSTRACT

The present invention relates to novel compounds capable of localized fluorescent signal amplification by peroxidase, a method for preparing them, and their uses as fluorescent probes. The compounds according to the present invention serve as new fluorescent amplification probes utilizing peroxidase, enabling the generation of a localized labeling pattern at the target site. Through this, they provide super-resolution protein localization information and allow the visualization of interactions between various organelles. Furthermore, they can provide localized and stable fluorescent signals in CLEM and super-resolution imaging and enable selective fluorescent labeling under fixed conditions, making them highly applicable in future proteomics and live-cell imaging.

### CROSS-REFERENCE TO RELATED APPLICATION

This application claims benefit from Korean Patent Application No. 10-2024-0009606, filed Jan. 22, 2024, the disclosure of which is incorporated herein in its entirety by reference, and priority is claimed.

### REFERENCE TO SEQUENCE LISTING

The instant application contains a Sequence Listing which has been submitted electronically in XML format and is hereby incorporated by reference in its entirety. Said XML copy, created on Jan. 21, 2025 is named “SOP116918US_Sequence Listing.xml” and is 1,770 bytes in size.

### TECHNICAL FIELD

The present invention relates to novel compounds capable of fluorescent signal amplification, a method for preparing the same, and uses thereof. More specifically, it relates to novel compounds that enable localized fluorescent signal amplification by peroxidase a method for preparing the same, and uses thereof as fluorescent probes.

### BACKGROUND

Correlative light and electron microscopy (CLEM) is an imaging approach capable of obtaining two distinct types of imaging information from the same sample. Fluorescence microscopy (FM) provides multicolor data on various proteins of interest (POIs), while electron microscopy (EM) reveals the ultrastructure of organelles. As a result, CLEM has become an indispensable tool in cell biology and neuroscience, enabling a comprehensive understanding of POIs and their connections with intracellular structures or subcellular compartments. For fluorescence-mediated visualization of POIs, the proteins are typically genetically fused with fluorescent proteins (FPs). However, the fluorescence from FPs in cells is often quenched during the harsh processes of EM sample preparation, specifically during dehydration, heating, and resin embedding. These steps can damage the protein structure and, consequently, its fluorescence (Choi, C. R., and Rhee, H. W. (2022). Proximity labeling: an enzymatic tool for spatial biology. Trends Biotechnol 40, 145-148). To overcome this challenge, fluorescence imaging may be conducted before the EM sampling process (Sharma, N. et al. (2023). A multifunctional peroxidase-based reaction for imaging, sensing and networking of spatial biology. Biochimica et Biophysica Acta (BBA)—Molecular Cell Research 1870, 119428). However, sequential imaging in this manner can lead to coordination issues between FM and EM images, as the localization of subcellular structures and proteins can change slightly during EM sampling. To overcome this issue, fluorescent reporters that can effectively retain fluorescence signals throughout the EM sample processing conditions are required to obtain distortion-free CLEM images.

Following the recent trend of CLEM imaging probe development, in this invention, a peroxidase-mediated fluorescence signal amplification approach has been developed to generate strong fluorescence signals useful for distortion-free CLEM imaging of proteins of interest (POIs). Fluorescence-based signal amplification utilizing peroxidase could provide a promising method to generate a large number of fluorescent radicals tagged to nearby biomolecules. In particular, if these tags can withstand the harsh sampling conditions of EM, they have potential applications in CLEM imaging. However, reported small-molecule fluorescent materials that can amplify fluorescence in the region of interest are still limited. Therefore, the exploration of fluorescence signal amplification via a peroxidase-based method could be a valuable approach for CLEM imaging.

Accordingly, the present inventor has made extensive efforts to develop a novel fluorescent probe capable of generating a strong fluorescent signal useful for CLEM imaging and has completed the present invention by synthesizing a novel compound capable of effectively amplifying a fluorescent signal at a localized site within a cell via peroxidase.

### SUMMARY OF THE INVENTION

The present invention aims to provide novel compounds that can be utilized as new fluorescent amplification probes designed for APEX labeling, a method for preparing the same, and uses thereof.

To achieve the above objective, the present invention provides a compound of the following Chemical Formula I or Chemical Formula II:

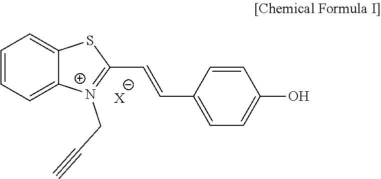

(X represents an atom or a molecular group that acts as a monovalent anion);

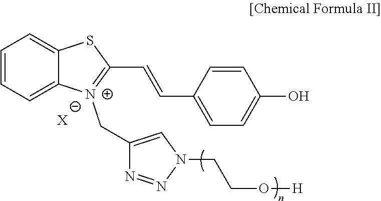

(n represents 1, 2, 3, 4, or 5, and X represents an atom or a molecular group that acts as a monovalent anion).

The present invention also provides a method for preparing the compound of Chemical Formula I, comprising:
(a) synthesizing (E)-2-(4-hydroxystyryl)-3-(prop-2-yn-1-yl)benzo[d]thiazol-3-ium through a Knoevenagel condensation reaction between 4-hydroxybenzaldehyde and 2-propargyl benzothiazolium salt.

The present invention also provides a method for preparing the compound of Chemical Formula II, comprising:
(a) synthesizing (E)-2-(4-hydroxystyryl)-3-(prop-2-yn-1-yl)benzo[d]thiazol-3-ium through a Knoevenagel condensation reaction between 4-hydroxybenzaldehyde and 2-propargyl benzothiazolium salt; and
(b) synthesizing the compound of Chemical Formula II by reacting (E)-2-(4-hydroxystyryl)-3-(prop-2-yn-1-yl)benzo[d]thiazol-3-ium with Azido-PEGn (where n is an integer from 1 to 5) through a Copper-Catalyzed Azide-Alkyne Cycloaddition (CuAAC) reaction.

In the present invention, wherein step (a) comprises adding a pyridine catalyst and stirring at a temperature of 50-60° C. for 8-16 hours to carry out the Knoevenagel condensation reaction.

In the present invention, wherein step (b) comprises adding NaHCO₃, sodium ascorbate, and CuSO₄·5H₂O, and stirring at room temperature for 20 minutes to 3 hours to carry out the Copper-Catalyzed Azide-Alkyne Cycloaddition (CuAAC) reaction.

In the present invention, wherein step (a) further comprises precipitation, filtration, washing, and/or separation after the reaction is completed to obtain the synthesized (E)-2-(4-hydroxystyryl)-3-(prop-2-yn-1-yl)benzo[d]thiazol-3-ium.

In the present invention, wherein step (b) further comprises extraction, drying, and/or purification after the reaction is completed to obtain the compound of Chemical Formula I.

The present invention also provides a fluorescent probe comprising the compound.

In the present invention, wherein the compound generates a fluorescence signal amplified by a peroxidase.

In the present invention, wherein the compound amplifies a localized fluorescence signal within a cell in vitro.

In the present invention, wherein the peroxidase is selected from the group consisting of APEX, APEX2, and HRP.

In the present invention, wherein the fluorescent probe is used for an electron microscopy or a correlative light and electron microscopy.

The present invention also provides a method for intracellular peroxidase-mediated fluorescence signal amplification, comprising incubating the fluorescent probe of claim 8 with hydrogen peroxide in cells overexpressing a peroxidase

In the present invention, wherein the compound in the fluorescent probe generates a fluorescence signal amplified by a peroxidase.

In the present invention, wherein the compound in the fluorescent probe amplifies a localized fluorescence signal within a cell in vitro.

In the present invention, wherein the peroxidase is selected from the group consisting of APEX, APEX2, and HRP.

In the present invention, wherein the fluorescent probe is used for an electron microscopy or a correlative light and electron microscopy.

In the present invention, wherein the fluorescent probe is applied at a concentration of 10-1000 μM, hydrogen peroxide (H₂O₂) is applied at a concentration of 0.1-5 mM, and the incubation is carried out for 1-10 minutes.

The present invention also provides a method for identifying an intracellular location of a protein of interest (POI), comprising:
(a) expressing a peroxidase fused with a protein of interest in a cell;
(b) incubating the cell after treatment with the fluorescent probe of claim 8 and hydrogen peroxide; and
(c) identifying a cellular organelle where amplified fluorescence signals from the fluorescent probe are observed.

The compounds according to the present invention are novel fluorescence amplification probes utilizing peroxidase. These probes maintain fluorescence even when treated with OsO₄, in contrast to existing probes. They can generate a localized labeling pattern at a target site, providing super-resolution protein location information and enabling the visualization of interactions between various organelles. Additionally, they offer local and stable fluorescence signals in CLEM and super-resolution imaging, and can implement selective fluorescence labeling under fixed conditions. As a result, they hold high potential for future applications in proteomics and live cell imaging.

### BRIEF DESCRIPTION OF DRAWINGS

FIG. 1A schematically illustrates APEX labeling, using biotin phenol, Amplex Red, and diaminobenzidine (DAB), for optical detection of POI-APEX2 localization.

FIG. 1B shows proposed model using the JFT1 probe for precipitate generation through the reaction of HRP and H₂O₂ (left) and model using only the probe and HRP (right).

FIG. 1C is a schematic illustrating the diverse applications of Jenfluor derivatives for live-cell APEX reactions, proteomics, and fixed-cell CLEM imaging.

FIG. 2A demonstrates the characterization of a diffusive or restricted TOM20-V5-APEX2-mediated outer mitochondrial membrane labeling pattern of Amplex Red, JFT1, and desthiobiotin-phenol (DBP). Regions labeled with DBP were visualized using Streptavidin-AF568, while probe-labeled regions were visualized using RFP channel. Anti-V5 antibody staining was detected with an anti-mouse-AF647 secondary antibody (Cy5 channel). Pearson correlation analysis utilized two-dimensional intensity histograms generated from Coloc2 analysis in Fiji/ImageJ. Scale bar, 10 μm.

FIG. 2B shows the line scan analysis results of the probe-labeled regions detected by the RFP channel and TOM20-V5-APEX2-expressed regions labeled by anti-V5, detected in Cy5 channel, in FIG. 2A.

FIG. 2C is a schematic illustrating two CLEM sampling procedures: post-embedding osmium treatment (upper) and pre-embedding osmium treatment (lower) after JFT1 labeling. POI indicates the protein of interest.

FIG. 2D presents a bar graph showing osmium-resistant fluorescence intensity of JF and JFT1 after 0.1% OsO₄ treatment.

FIG. 2E shows CLEM imaging of the JFT1-labeled mitochondrial matrix regions by Matrix-V5-APEX2. This sample was obtained by following the pre-embedding osmium treatment procedure in FIG. 2C. For JFT1 labeling, JFT1 (500 μM) and 1 mM H₂O₂ were applied to the fixed cells expressing matrix-V5-APEX2 for 5 minutes. JFT1 fluorescence was detected in the RFP channel of the fluorescence microscope. Scanning electron microscopy (SEM) was used for EM imaging. Black scale bar, 10 μm.

FIG. 3 shows confocal imaging results of intracellular FLEX staining using Jenfluor-triazole (JFT) for various organelle-targeted APEX2-POIs [POI=NLS (nucleus), SEC61B (ER membrane), PLIN2 (lipid droplet), CD63 (endolysosomal membrane), TOM20 (outer mitochondrial membrane), TMEM192 (lysosomal membrane)]. Detailed information of APEX2-POI constructs is provided in Table 1. For APEX2 labeling in fixed cell condition, JFT1 (100 μM) were treated for 5 minutes with 1 mM H₂O₂ in 100 mM PHEMS buffer. JFT1-labeled regions were visualized with GFP and RFP channels. Anti-V5 antibody staining was detected with anti-mouse-AF647 secondary antibody (Cy5 channel). Scale bar, 10 μm.

FIG. 4A shows CLEM imaging of the JFT1-labeled region by CD63-V5-APEX2. Scale bars, 10 μm and 500 nm.

FIG. 4B shows CLEM imaging of the JFT1-labeled region by APEX2-V5-PLIN2. Red arrows indicate sites of contact between mitochondria and the endoplasmic reticulum (ER). Scale bar, 20 μm.

FIG. 4C shows CLEM imaging of the JFT1-labeled region by SEC61B-V5-APEX2. JFT1 was subjected to en bloc staining before and after embedding. Fluorescence was registered post-embedding, followed by TEM imaging. Red arrows indicate well-preserved ER tubules. Scale bar, 5 μm.

FIG. 5A shows confocal imaging results of JFT1-labeled region by TMEM192-V5-APEX2 in control or Bafilomycin A1 (BFA)-treated cells. JFT1-labeled region was detected in GFP and RFP channels and anti-V5 antibody staining was detected with anti-mouse-AF647 secondary antibody (Cy5 channel). Scale bar, 10 μm.

FIG. 5B shows quantification of the lysosomal diameter (JFT1-labeled region by TMEM192-V5-APEX2) under the DMSO and BFA (100 nM, overnight)-treated sample (n=30 of lysosome). Data are presented as mean±SEM.

FIG. 5C shows two-color CLEM imaging of BFA-treated mito-mEosEM (GFP channel) and TMEM192-V5-APEX2 (JFT1-labeled, RFP channel) expressed cells. In this image, green fluorescence signal of JFT1 was negligible compared to that of Mito-mEosEM. Mito-mEosEM was mainly detected in the GFP channel. Red arrows denote mitochondria in lysosomes. Scale bar, 1 μm.

FIG. 5D shows two-color CLEM imaging of mito-mEosEM (GFP channel) and TMEM192-V5-APEX2 (JFT1-labeled, RFP channel) expressed cells under the U18666A treatment condition (2 μg/ml, overnight). Blue arrows denote mitochondria-lysosomes contact sites. Scale bar, 1 μm.

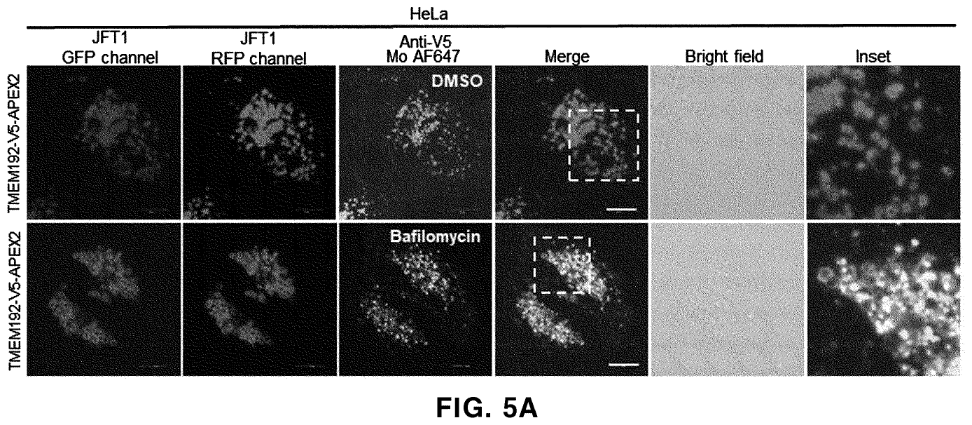

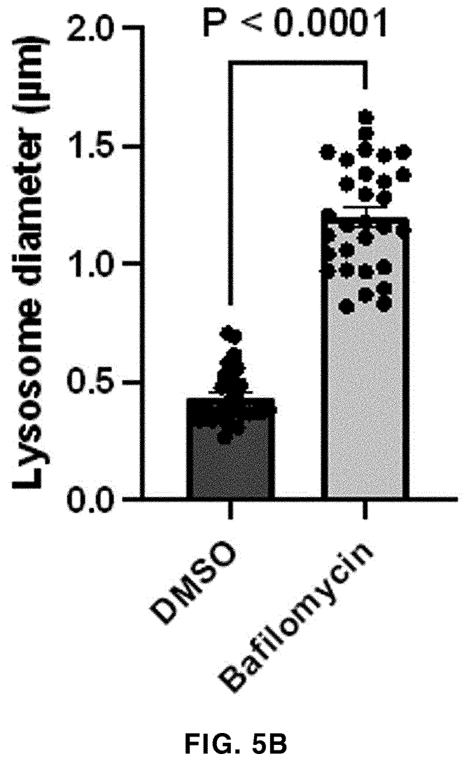

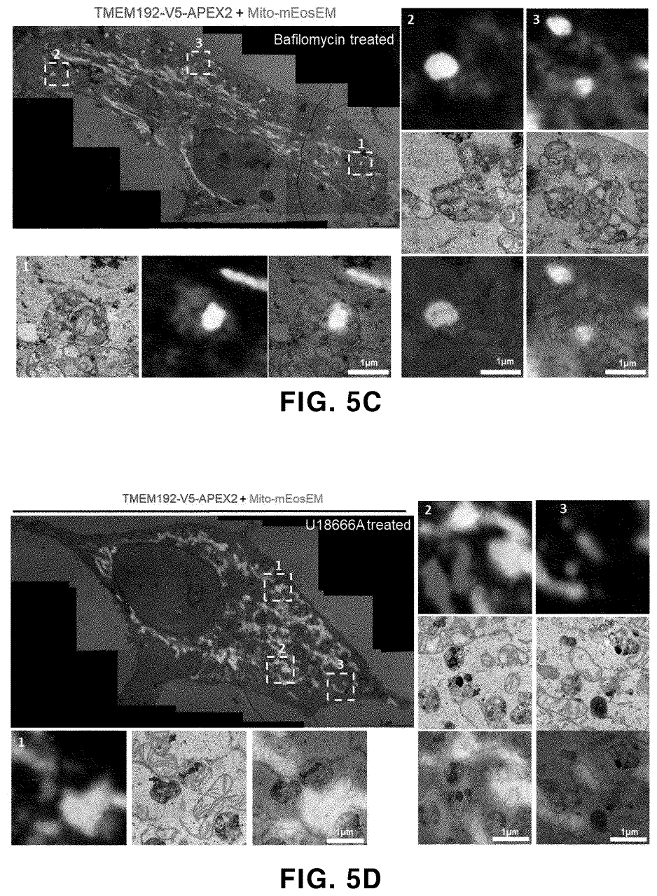

For reference, FIGS. 5C and 5D were performed with confocal imaging after JFT1 labeling and cells were processed for EM imaging following the post-embedding process.

### DETAILED DESCRIPTION

Unless otherwise defined, all technical and scientific terms used herein have the same meaning as commonly understood by one of ordinary skill in the art to which this invention belongs. In general, the nomenclature used herein is well known and commonly used in the art.

In the concentration range described herein, “to” is used to mean including (both above and below) both critical ranges, and when both critical ranges are not included, the concentration range is described as “exceeding” and “less than”. The term “about” used in a numerical value herein is used to mean including a range that is expected to exhibit substantially the same effect as the numerical value described by those skilled in the art, and may be, for example, ±20%, ±10%, ±5%, etc. of the described numerical value, but is not limited thereto.

Fluorescent tagging of biomolecules enables their sensitive detection during separation and determining their subcellular location. In this context, peroxidase-based reactions are actively utilized for signal amplification. To harness this potential, a genetically encodable enzymatic fluorescence signal amplification method using APEX (FLEX) was developed in the present invention. In that, in the present invention, a fluorescent probe, Jenfluor triazole (JFT1), was newly synthesized, which effectively amplifies and restricts fluorescence signals under fixed conditions, enabling fluorescence-based detection of subcellularly localized, electron-rich metabolites. Moreover, JFT1 exhibited stable fluorescence signals even under osmium-treated and polymer-embedded conditions, which supported findings from correlative light and electron microscopy (CLEM) using APEX. Using various APEX-conjugated proteins of interest (POIs) targeted to different organelles, their localization was successfully visualized through FLEX imaging while effectively preserving organelle ultrastructures. FLEX provides insights into dynamic lysosome mitochondria interactions upon exposure to chemical stressors. Overall, it was confirmed that FLEX holds significant promise as a sensitive and versatile system for fluorescently detecting APEX2-POIs in multiscale biological samples in the present invention.

### Novel Compounds and Preparing Method Thereof

Accordingly, in one aspect, the present invention relates to a compound of Chemical Formula I or Chemical Formula II, and a method for preparing the same.

A compound of Chemical Formula I or Chemical Formula II:

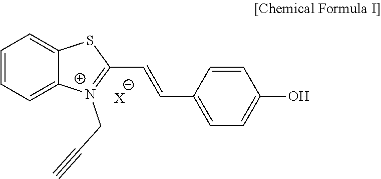

(In Chemical Formula I, X represents an atom or a molecular group that can act as a monovalent anion.)

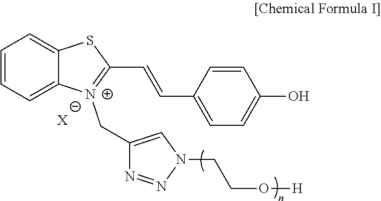

(In Chemical Formula II, n represents 0, 1, 2, 3, 4, or 5, and X represents an atom or molecular group that can act as a monovalent anion.)

In the compound of Chemical Formula I or Chemical Formula II of the present invention, X⁻ is not limited thereto but may be a monovalent anion such as F⁻, Cl⁻, Br⁻, I⁻, NO₃⁻, or ClO₄⁻.

In the present invention, the chemical formula may represent a compound having the structure depicted by the structural formula and its specific modifications or forms. In particular, the compound of the chemical formula provided herein may have an asymmetric center, so it may exist in different enantiomeric forms. All optical isomers of the compound of the general chemical formula and their mixtures are considered to fall within the scope of the chemical formula. Therefore, any chemical formula provided herein is intended to represent racemates, one or more enantiomeric forms, one or more diastereomeric forms, one or more atropisomeric forms, and mixtures thereof. Additionally, specific structures may exist as geometric isomers (i.e., cis and trans isomers), tautomers, or atropisomers.

The term “isomer” in this specification refers to compounds that have the same molecular formula but differ in the nature or sequence of atomic bonds or the arrangement of atoms in space. Stereoisomers that are not mirror images of each other are referred to as “diastereomers,” while mirror-image isomers are referred to as “enantiomers.” For example, if a compound has an asymmetric center, it can be bonded to four different substituents, allowing for the possibility of a pair of enantiomers. Enantiomers can be characterized by the absolute arrangement of the asymmetric center, described by the R- and S-sequencing rules of Cahn and Prelog, or by the way the molecule rotates the plane of polarized light and is designated as either (+)- or (−)-isomers. Chiral compounds can exist as individual enantiomers or as mixtures thereof. A mixture containing equal amounts of both enantiomers is referred to as a “racemic mixture.” “Tautomers” refer to interconvertible forms of a compound structure, where the displacement of hydrogen atoms and electrons results in different compounds. Thus, the two structures can exist in equilibrium through the movement of electrons and atoms (usually hydrogen). An example of tautomers is the enol and keto forms, which rapidly interconvert under acidic or basic conditions. Another example of tautomers includes the acidic and nitro forms of phenyl nitromethane, which also interconvert under acidic or basic conditions. The existence of tautomers may be related to the achievement of optimal chemical reactivity and biological activity of the compound of interest.

The compounds of the present invention may have one or more asymmetric centers; therefore, such compounds can be generated as individual (R)- or (S)-stereoisomers or as mixtures thereof. Unless otherwise indicated, the description or naming of specific compounds in the specification and claims is intended to include both individual enantiomers and their mixtures (racemic mixtures or others). Methods of determining stereochemistry and separating stereoisomers are well-known in the art.

Any chemical formula of the present invention, even if its form is not explicitly listed, refers to hydrates, solvates, polymorphs, and mixtures thereof. Compounds of Formula I or Formula II, or pharmaceutically acceptable salts of the compounds of Formula I or Formula II, may be obtained as solvates. Solvates include those formed from the interaction or complexation of the compound of the present invention with one or more solvents, either in solution or in solid or crystalline form.

In another aspect, the present invention provides a method for preparing the compound of Chemical Formula I or the compound of Chemical Formula II.

The method for preparing the compound of Chemical Formula I may comprise:
(a) synthesizing (E)-2-(4-hydroxystyryl)-3-(prop-2-yn-1-yl)benzo[d]thiazol-3-ium through a Knoevenagel condensation reaction between 4-hydroxybenzaldehyde and 2-propargyl benzothiazolium salt.

The method for preparing the compound of Chemical Formula II may comprise:
(a) synthesizing (E)-2-(4-hydroxystyryl)-3-(prop-2-yn-1-yl)benzo[d]thiazol-3-ium through a Knoevenagel condensation reaction between 4-hydroxybenzaldehyde and 2-propargyl benzothiazolium salt; and
(b) synthesizing the compound of Chemical Formula II by reacting (E)-2-(4-hydroxystyryl)-3-(prop-2-yn-1-yl)benzo[d]thiazol-3-ium with Azido-PEGn (where n is an integer from 1 to 5) through a Copper-Catalyzed Azide-Alkyne Cycloaddition (CuAAC) reaction.

In the present invention, the Knoevenagel condensation reaction of step (a) forms an α,β-unsaturated compound between 4-hydroxybenzaldehyde and 2-propargyl benzothiazolium salt, and at this time, pyridine acts as a base catalyst to induce activation of the aldehyde and a condensation reaction with the propargyl benzothiazolium salt, thereby synthesizing (E)-2-(4-hydroxystyryl)-3-(prop-2-yn-1-yl)benzo[d]thiazol-3-ium (Alkyne-Jenfluor (JFA)).

In the step (a), 4-hydroxybenzaldehyde is dissolved in alcohol (e.g., methanol), 2-propargyl benzothiazolium salt is added, and further stirred at room temperature, after which pyridine can be added as a catalyst. Thereafter, the Knoevenagel condensation reaction can be induced by stirring at about 50 to about 60° C. for about 8 to about 16 hours. When the reaction is completed, the reaction mixture can be cooled to room temperature and concentrated in vacuum to obtain a dark red crude. Thereafter, ethyl acetate (10 mL) can be added to the dark red crude to precipitate the product. The generated solid can be recovered by vacuum filtration and further washed. In this case, the washing can be performed three or more times with ethyl acetate.

Thereafter, the generated (E)-2-(4-hydroxystyryl)-3-(prop-2-yn-1-yl)benzo[d]thiazol-3-ium (Alkyne-Jenfluor (JFA)) can be separated in the form of a dark red powder using a Buchner funnel or the like.

The (E)-2-(4-hydroxystyryl)-3-(prop-2-yn-1-yl)benzo[d]thiazol-3-ium (Alkyne-Jenfluor (JFA)) synthesized in this manner is an unsaturated compound containing a propargyl group, which can be utilized as a reactant for further chemical modification (e.g., click reaction, etc.) in step (b).

In the present invention, the Copper-Catalyzed Azide-Alkyne Cycloaddition (CuAAC) reaction in step (b) forms a 1,2,3-triazole ring through a click reaction between an alkyne group (—C≡C) and an azide group (—N₃), and at this time, a copper (I) complex (Cu+) acts as a catalyst to synthesize a compound of chemical formula II.

In the step (b), (E)-2-(4-hydroxystyryl)-3-(prop-2-yn-1-yl)benzo[d]thiazol-3-ium (Alkyne-Jenfluor (JFA)) synthesized in the step (a) is dissolved in acetonitrile together with Azido-PEGn (n is an integer from 1 to 5), and NaHCO₃, sodium ascorbate, and CuSO₄·5H₂O as catalysts and auxiliary agents are sequentially added in water, and the mixture is stirred at room temperature for about 20 minutes to about 3 hours to induce high-efficiency coupling between JFA and Azido-PEGn using click chemistry, thereby synthesizing the compound of chemical formula II. After the reaction, the organic layer can be extracted with isopropanol/chloroform, and a drying step can be performed in which residual moisture is removed through a Na₂SO₄ pad, and the solvent is removed under reduced pressure. Thereafter, the compound of formula II can be obtained as a crystalline solid using silica gel column chromatography (about 8-10% methanol/chloroform).

### Fluorescent Probe

In another aspect, the present invention relates to the use of the compound of formula I or formula II as a fluorescent probe.

In the present invention, the compound may be characterized by generating a fluorescent signal amplified by peroxidase.

In the present invention, the compound may be characterized by amplifying a fluorescent signal localized within a cell in vitro.

In the present invention, the peroxidase may be characterized by being selected from the group consisting of APEX (Ascorbate Peroxidase), APEX2, and HRP (Horseradish Peroxidase), but is not limited thereto.

The HRP may be in a form bound to an antibody (antibody-HRP).

In the present invention, the fluorescent probe may be characterized as a fluorescent probe for an electron microscope or a correlative light and electron microscopy, but is not limited thereto.

The fluorescent probe according to the present invention can amplify a fluorescent signal within a cell by peroxidase.

In another aspect, the present invention provides a method for amplifying a cellular peroxidase-mediated fluorescence signal, comprising the step of treating a cell in which peroxidase is overexpressed with the fluorescent probe and hydrogen peroxide and incubating the same.

In the present invention, the fluorescent probe may be treated at about 10 to about 1000 uM and the hydrogen peroxide (H₂O₂) at about 0.1 to about 5 mM and incubating for about 1 to about 10 minutes.

In another aspect, the present invention provides a method for identifying a cellular location of a protein of interest (POI), comprising:
(a) expressing a fusion of peroxidase with a protein of interest in a cell;
(b) incubating the cell after treatment with the fluorescent probe of claim 8 and hydrogen peroxide; and
(c) identifying a cellular organelle where amplified fluorescence signals from the fluorescent probe are observed. In the present invention, the cell may be characterized as being an in vitro cell.

In the present invention, the proximity labeling enzyme APEX has been identified as a highly valuable tool for the peroxidase signal amplification strategy, given its ability to catalytically generate phenoxy radicals that covalently attach to proximal tyrosine-containing biomolecules within a defined space. Genetically encodable peroxidases such as horseradish peroxidase (HRP) and APEX2 have found widespread use in spatial proteomics and electron microscopy (EM) workflows. In spatial proteomics applications, biotin-phenol or alkyne-phenol probes are commonly employed: however, these probes lack fluorescence and result in diffusive labeling patterns, even under fixed conditions. For APEX-EM imaging applications, diaminobenzidine (DAB) generates a restricted DAB polymer stain at APEX-expressing sites. Unfortunately, the DAB stain lacks fluorescence, and its dark stain can only be observed in bright-field imaging from an APEX-generated DAB polymer. Currently, Amplex Red remains the only fluorogenic substrate routinely used for peroxidase labeling. However, its oxidized product, resorufin, is both stable and diffusible from the site of peroxidase reaction.

In the present invention, a novel fluorescence amplification probe designed for APEX labeling has been developed, and it has been confirmed that the probe can produce a localized fluorescent stain ideal for APEX-CLEM. The probe was found to amplify fluorescence signals in specific regions of interest and can then be used for subsequent CLEM analysis as a fluorescent peroxidase substrate.

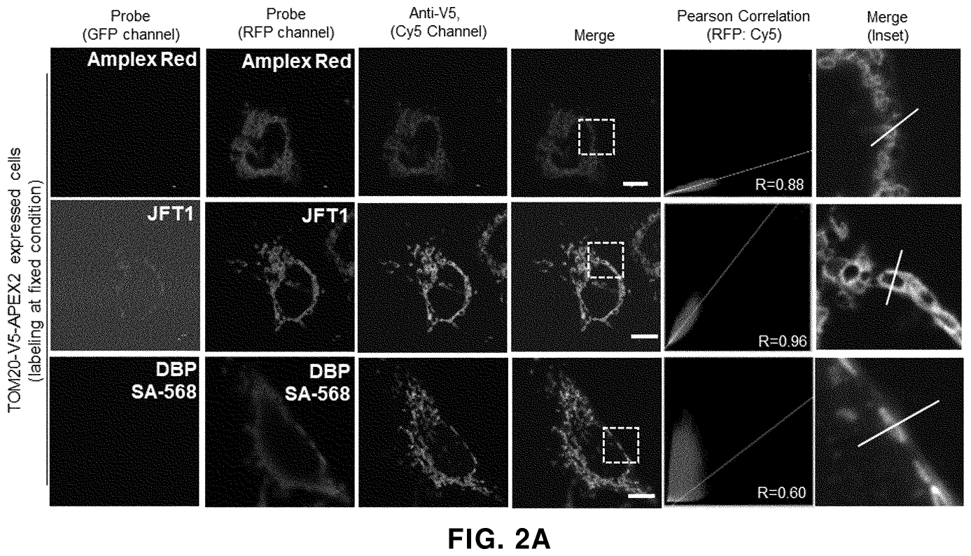

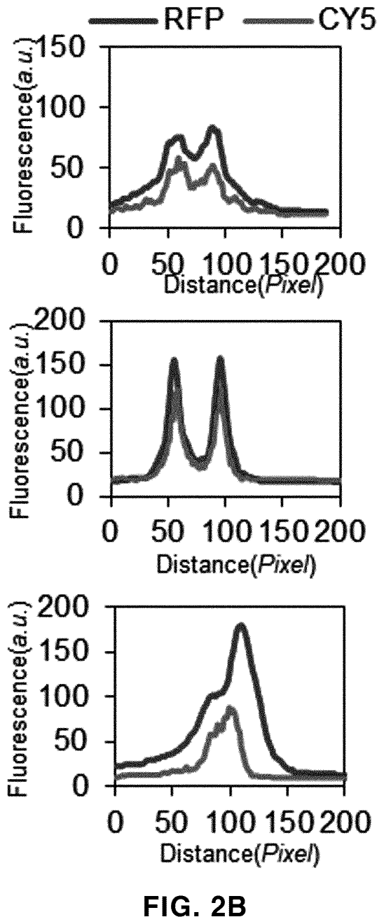

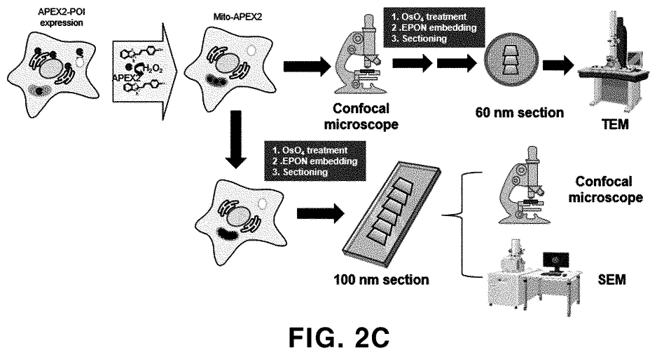

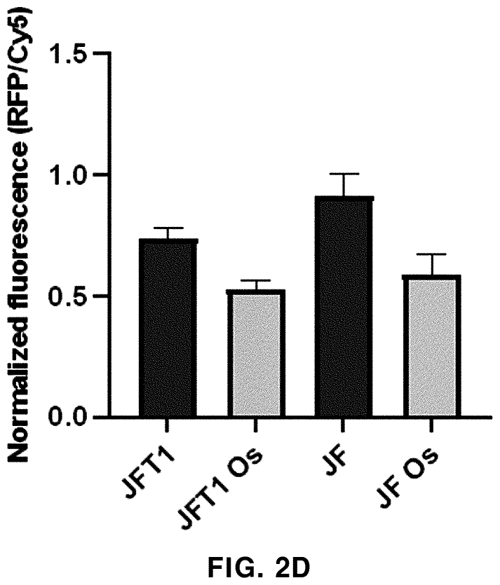

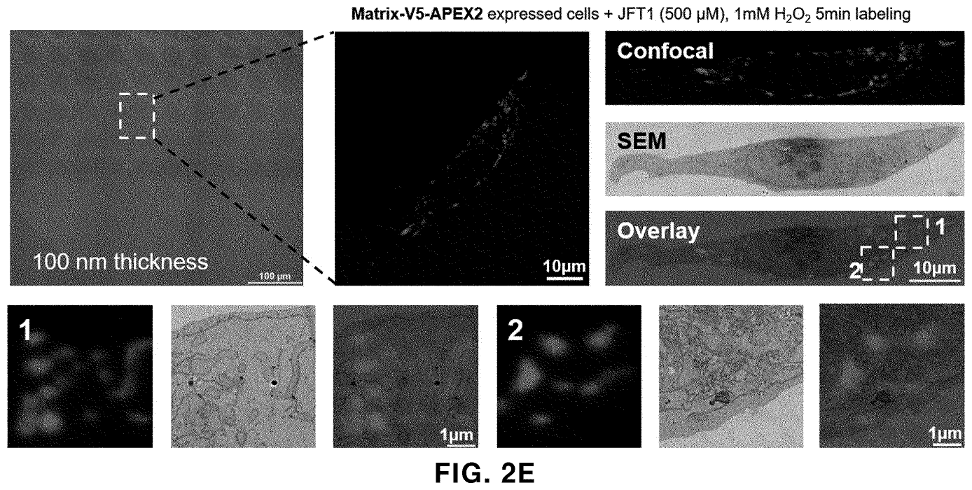

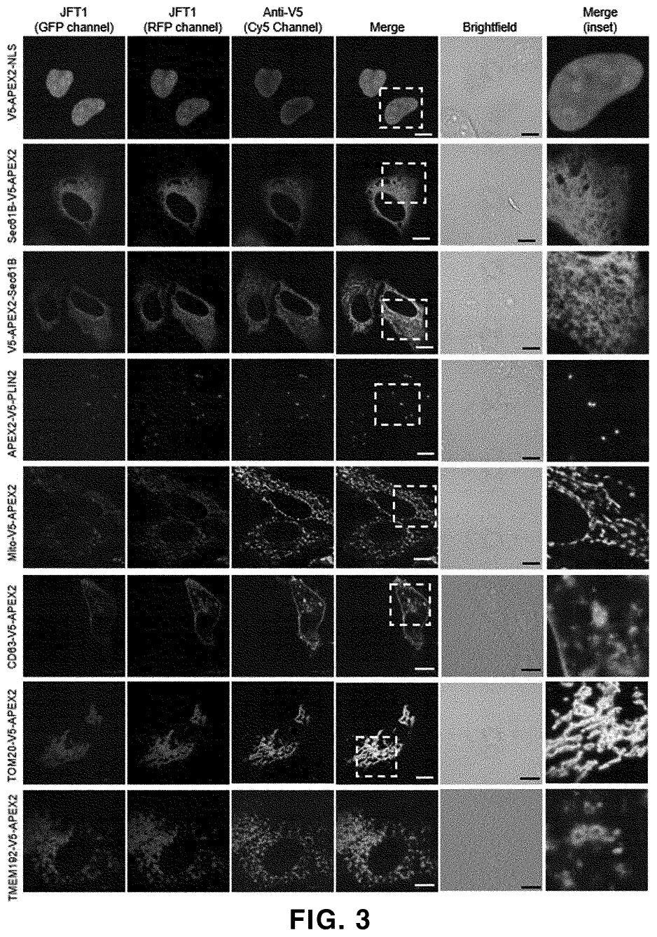

In the present invention, we developed a fluorescent probe (JFT1), which exhibits a fluorescence-amplified signal when APEX is expressed. Unlike conventional fluorophores or fluorescent proteins that suffer from loss of fluorescence during pre-embedding OsO₄ treatment, JFT1 generates a well-retained and restricted fluorescence pattern in both pre-embedding OsO₄-treated samples (FIG. 2E) and post-embedding OsO₄-treated samples (FIG. 5). The well-preserved fluorescence signal of JFT1 holds promise for revealing super-resolution localization information of POIs in various organelles (FIG. 3). The mechanism underlying our probe is akin to the tyramide signal amplification (TSA) mechanism, which involves accumulated fluorescent phenoxy radical labeling by targeted peroxidase. In contrast to normal TSA probes or biotin-phenol probes that exhibit diffusive labeling patterns, JFT1 exhibits a restricted labeling pattern due to its stilbene structure. This structure enables the ethylene moiety to participate in the polymerization reaction in the radical state, similar to the polymerization reaction of other stilbene derivatives. Likewise, DAB is known to form polymers as its radicals induce polymerization reactions. We observed the formation of the polymerized product of JFT in a test-tube reaction with HRP, and the heterogeneous properties of JFT during APEX staining (e.g., green fluorescence under neutral pH) support its heterogeneous polymerization mode.

Meanwhile, the chemical synthesis of JFT was facilitated through a three-step reaction involving sample and robust chemical processes (overall product yield ˜60%). Notably, JFT probes displayed improved solubility compared to the original JF probe in aqueous solutions, ensuring reliable imaging results. Based on these advantages, we propose that JFT holds potential as a versatile TSA probe or a fluorescent analog of the DAB molecule for APEX2-expressing samples as well as antibody-HRP-incubated TSA samples (Hernandez et al., 2021 Hernandez et al., (2021), Front. Mol. Biosci. 8, 66706).

Additionally, the present invention presents a method that combines the FLEX labeling strategy with the mEosEM protein for two colored CLEM to generate dynamic snapshots of the interactions between the mitochondria and lysosomes. Compared to the super-resolution microscope imaging techniques that can selectively visualize proteins or regions of interest with fluorescent tags, FLEX labeling with CLEM offers the advantage of visualizing not only the fluorescent region of interest but also its interactions with the non-fluorescent tagged surrounding structures. Furthermore, the FLEX method demonstrates membrane preservation as evidenced from our APEX2-V5-PLIN2 and SEC61B-V5-APEX2 CLEM experiments, conforming a well-preserved membrane morphology and spatially restricted fluorescence amplification. The broad applicability of the FLEX-CLEM approach in targeting various organelles simultaneously and observing them using different imaging platforms offers an ideal tool for CLEM. Furthermore, our FLEX method can be enhanced by integrating it with super-resolution imaging techniques, provided that our current probe or another version of the FLEX will be compatible with super-resolution imaging modalities.

However, our focus was on utilizing JFT for CLEM imaging under fixed conditions. For live-cell APEX2 labeling experiments, JF or JFA (50-100 μM) was incubated with 1 mM H₂O₂ for 1 min. Both JF and JFA are cell-permeable and can react with APEX2 under live-cell conditions. However, it is important to note that JF and JFA can also react with endogenous peroxidases when incubated at high concentrations (≥100 μM) for 1 h in live cells. In contrast, under fixed-cell conditions, the endogenous peroxidase activity was found to be quenched. We observed that a 5-min incubation of high concentrations of JFT1 (100-500 M) and H₂O₂ (0.5-1 mM) can generate selective FLEX labeling where APEX2-POIs are expressed (see Methods for details). For live-cell experiments, it would be highly advantageous to further develop JF probes with negligible activity against endogenous peroxidase in live-cell conditions while still maintaining reactivity for the APEX or HRP reaction. Since JFT was synthesized from JFA, this strategy opens up the possibility of generating further variations in JFT or other JF-based probes using various azido-click moieties. This approach could lead to the development of improved probes with enhanced performance for live-cell imaging applications.

In the present invention, JFA-labeled proteins can be modified with biotin-azide using a copper-click reaction. However, we did not analyze the modified proteins after enrichment using streptavidin-beads in this work. Previous research has shown the successful use of alkynyl-based probes, similar to JFA, as APEX probes in spatial proteomics studies (Li et al., 2020; Qin et al., 2023). This suggests that JFA has the potential to be employed in future proteomics analysis approaches.

### EXAMPLES

Hereinafter, the present invention will be described in more detail through examples. These examples are only for illustrating the present invention, and it will be obvious to those skilled in the art that the scope of the present invention is not limited by these examples.

### Example 1

### Chemical Synthesis

### 1-1. Materials and General Methods

All nuclear magnetic resonance (NMR) experiments were carried out using a Varian NMR spectrometer equipped with Shield 500 MHz Oxford magnet (AS500)/5 mm ID PFG. NMR spectrum optimization was conducted using Mnova software. The compounds were dissolved in DMSO-d₆ and the spectra were acquired at 25° C. Column chromatography was performed using silica. Starting materials were obtained from Sigma-Aldrich (St. Louis, MO, USA), or Alfa Aesar (Ward Hill, MA, USA). Solvents were obtained from Fisher Scientific (Hampton, NH, USA) or Sigma-Aldrich without further purification.

### 1-2. Synthesis of JF, JFA and JFT

### (E)-3-ethyl-2-(4-hydroxystyryl)benzo[d]thiazol-3-ium (Jenfluor; JF) Synthesis

In a flask, 1.1 mmol of the 4-hydroxybenzaldehyde was dissolved in 25 mL of methanol. Then, the 2-ethyl benzothiazolium salt (R+X; 1.0 mmol) was added and the solution was stirred at room temperature for 10 minutes. Following the addition of pyridine (0.25 mL), the resulting solution was heated up and stirred at 65° C. for 12 hours. After completion of the reaction, the reaction mixture was cooled down to room temperature and concentrated under vacuum. To the resulting dark red crude, ethyl acetate (10 mL) was added, and the product was precipitated as a dark colored solid in the bottom of the flask. After the solution was allowed to settle for 15 minutes, the resulting solid was collected by vacuum filtration and washed with ethyl acetate (10 mL) 3 times. The Jenfluor was collected using a Buchner funnel as deep red colored powder and characterization data were collected which matches with reported literature.

¹H NMR (500 MHz, DMSO-d₆): δ 10.63 (s, 1H), 8.40 (d, J=8.0 Hz, 1H), 8.25 (d, J=8.4 Hz, 1H), 8.17 (d, J=15.6 Hz, 1H), 7.98 (d, J=8.5 Hz, 2H), 7.88-7.83 (m, 1H), 7.81 (d, J=15.7 Hz, 1H), 7.76 (t, J=7.6 Hz, 1H), 6.94 (d, J=8.5 Hz, 2H), 4.92 (dd, J=13.9, 6.8 Hz, 2H), 1.45 (t, J=7.1 Hz, 3H).

### (E)-2-(4-hydroxystyryl)-3-(prop-2-yn-1-yl)benzo[d]thiazol-3-ium (Alkyne-jenfluor; JFA) Synthesis

In a flask, 1.1 mmol of the 4-hydroxybenzaldehyde was dissolved in 25 mL of methanol. Then, the 2-propargyl benzothiazolium salt (R+X; 1.0 mmol) was added and the solution was stirred at room temperature for 10 minutes. Following the addition of pyridine (0.25 mL), the resulting solution was heated up and stirred at 65° C. for 12 hours. After completion of the reaction, the reaction mixture was cooled down to room temperature and concentrated under vacuum. To the resulting dark red crude, ethyl acetate (10 mL) was added, and the product was precipitated as a dark colored solid in the bottom of the flask. After the solution was allowed to settle for 15 minutes, the resulting solid was collected by vacuum filtration and washed with ethyl acetate (10 mL) 3 times. The Jenfluor was collected using a Buchner funnel as deep red coloured powder and characterization data were collected which matches with reported literature.

¹H NMR (500 MHz, DMSO-d₆): δ 10.73 (s, 1H), 8.41 (d, J=7.9 Hz, 1H), 8.25 (d, J=3.7 Hz, 1H), 8.23 (d, J=2.0 Hz, 1H), 7.98 (d, J=8.6 Hz, 2H), 7.92-7.85 (m, 2H), 7.77 (t, J=7.8 Hz, 1H), 6.95 (d, J=8.5 Hz, 2H), 5.87 (s, 2H), 3.77 (s, 1H).

### General Synthesis Protocol for JFTs:

JFA (0.91 mmol) and Azido-PEGn (1.09 mmol) were dissolved in acetonitrile (14 mL), NaHCO₃ (191 mg, 2.28 mmol), sodium ascorbate (45 mg, 0.2 mmol), CuSO₄·5H₂O (57 mg, 0.2 mmol) was dissolved in water (7 mL) and added sequentially. There action mixture was stirred at room temperature for 1 hour. The organic layer obtained by extraction with isopropanol/chloroform was passed through a Na₂SO₄ Pad to remove remaining moisture, and then the solvent was removed under reduced pressure. Product was isolated using Silica-gel column chromatography (8-10% methanol/chloroform) to obtain JFT-PEGn as a crystalline solid.

### (E)-3-((1-(2-hydroxyethyl)-1H-1,2,3-triazol-4-yl)methyl)-2-(4-hydroxystyryl)benzo[d]thiazol-3-ium (JFT-PEG1, JFT1)

¹H NMR (500 MHz, DMSO-d₆) δ 8.29 (s, 1H), 8.05 (d, J=7.8 Hz, 1H), 7.99 (d, J=8.1 Hz, 1H), 7.77-7.68 (m, 3H), 7.62 (t, J=7.5 Hz, 1H), 7.47 (t, J=7.5 Hz, 1H), 7.38 (d, J=14.2 Hz, 1H), 6.47 (d, J=8.3 Hz, 2H), 5.89 (s, 2H), 4.39 (s, 2H), 3.73 (s, 2H). ¹³C NMR (125 MHz, DMSO-d₆) δ 190.88 (s), 166.48 (s), 148.29 (s), 141.21 (s), 139.96 (s), 135.06 (s), 128.18 (s), 125.77 (s), 125.50 (s), 124.66 (s), 123.25 (s), 121.43 (s), 114.50 (s), 100.37 (s), 59.73 (s), 52.34 (s), 41.62 (s). MS (ESI+) m/z 379.1207 [M]+.

### (E)-3-((1-(2-(2-hydroxyethoxy)ethyl)-1H-1,2,3-triazol-4-yl)methyl)-2-(4-hydroxystyryl)benzo[d]thiazol-3-ium (JFT-PEG2, JFT2)

¹H NMR (500 MHz, DMSO-d₆) δ 8.27 (s, 1H), 7.87 (d, J=7.8 Hz, 1H), 7.75 (d, J=8.3 Hz, 1H), 7.48 (t, J=7.5 Hz, 1H), 7.44 (d, J=13.5 Hz, 1H), 7.31 (t, J=7.6 Hz, 1H), 6.96 (d, J=13.5 Hz, 1H), 6.18 (d, J=8.6 Hz, 2H), 5.67 (s, 2H), 4.51 (t, J=5.1 Hz, 2H), 3.77 (t, J=5.1 Hz, 2H), 3.41 (m, 4H). ¹³C NMR (125 MHz, DMSO-d₆) δ 183.25 (s), 162.76 (s), 141.37 (s), 140.54 (s), 127.53 (s), 124.49 (s), 124.37 (s), 124.36 (s), 122.68 (s), 119.36 (s), 113.11 (s), 94.84 (s), 72.08 (s), 68.65 (s), 64.93 (s), 60.09 (s), 49.59 (s), 40.61 (s), 15.18 (s). MS (ESI+) m/z 423.1468 [M]+.

### (E)-3-((1-(2-(2-(2-hydroxyethoxy) ethoxy)ethyl)-1H-1,2,3-triazol-4-yl)methyl)-2-(4-hydroxystyryl)benzo[d]thiazol-3-ium (JFT-PEG3, JFT3)

¹H NMR (500 MHz, DMSO-d₆) δ 8.39 (s, 1H), 8.23 (d, J=7.9 Hz, 1H), 8.18 (d, J=8.4 Hz, 1H), 7.98 (d, J=14.9 Hz, 1H), 7.86 (d, J=8.6 Hz, 2H), 7.73 (dd, J=19.6, 11.6 Hz, 2H), 7.60 (t, J=7.6 Hz, 1H), 6.74 (d, J=8.5 Hz, 2H), 6.11 (s, 2H), 4.53-4.47 (m, 2H), 3.78-3.74 (m, 2H), 3.47-3.43 (m, 4H), 3.40 (d, J=4.7 Hz, 2H), 3.33 (t, J=5.0 Hz, 2H). ¹³C NMR (125 MHz, DMSO-d₆) δ 211.08 (s), 194.05 (s), 141.11 (s), 139.84 (s), 133.93 (d, J=2.1 Hz), 128.71 (s), 126.95 (s), 126.51 (s), 124.86 (s), 123.81 (s), 123.37 (s), 118.79 (s), 115.90 (s), 115.67 (s), 72.29 (s), 69.58 (s), 69.54 (s), 68.63 (s), 60.17 (s), 49.62 (s), 42.50 (s). MS (ESI+) m/z 467.1726 [M]+.

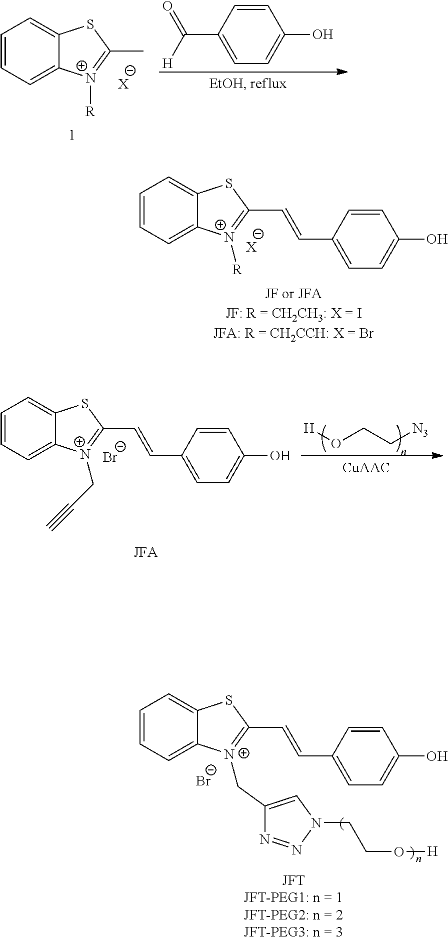

### Example 2

### Experimental Method

### 2-1. Cloning of Expression Plasmids

Genes were cloned into the specified vectors using standard enzymatic restriction digestion, and then ligated with T4 DNA ligase. To generate constructs in which short tags (e.g., V5 epitope tag) or signal sequences were appended to the protein, the tag was included in the primers used to PCR amplify the gene. The PCR products were then digested with restriction enzymes and ligated into cut vectors (e.g., pcDNA3 and pcDNA5). In all cases, the cytomegalovirus (CMV) promoter was used for expression in cells. The genetic constructs used in this study are listed in Table 1.

TABLE 1

| | | | Details | |
|---|---|---|---|---|
| Name | Features | Promoter/vector | (Localization) | Note |
| V5-APEX2-NES | KpnI-V5- | CMV-F/pCDNA5 | NES(Nuclear Export | Used in PMID: |
| | APEX2-NES- | | Signal) V5: | 36265183 |
| | STOP-NotI | | GKPIPNPLLGLDST | |
| | | | (SEQ ID NO: 1) | |
| Sec61B-V5- APEX2 | NotI-Sec61B-V5- | pCDNA3 | ER cytosol V5: | Used in PMID: |
| | APEX2-STOP | | GKPIPNPLLGLDST | 27184847 |
| | XhoI | | (SEQ ID NO: 1) | |
| APEX2-V5- PLIN2 | AfIII-APEX2- | CMV-F/pCDNA5 | Lipid Droplet V5: | Used in PMID: |
| | V5-PLIN2-Stop- | | GKPIPNPLLGLDST | 36265183 |
| | XhoI | | (SEQ ID NO: 1) | |
| V5-APEX2-Sec61B | AfIII-V5- | CMV-F/pCDNA5 | ER lumen V5: | Used in PMID: |
| | APEX2-Sec61B- | | GKPIPNPLLGLDST | 27184847 |
| | stop XhoI | | (SEQ ID NO: 1) | |
| V5-APEX2-NLS | AfIII-V5- | CMV-F/pCDNA5 | NLS(Nuclear | Used in PMID: |
| | APEX2-NLS- | | Localization Signal) | 27184847 |
| | Stop-XhoI | | V5: | |
| | | | GKPIPNPLLGLDST | |
| | | | (SEQ ID NO: 1) | |
| TMEM192-V5-APEX2 | KpnI-TMEM192- | CMV-F/pCDNA5 | Lysosomal membrane | Used as |
| | BamHI-V5 | | localization V5: | TMEM192- |
| | APEX2-NotI | | GKPIPNPLLGLDST | HA in PMID: |
| | | | (SEQ ID NO: 1) | 31995728 |
| Mito-mEosEM | NheI-MTS- | Purchased | Mitochondria matrix. | |
| | mEos4b-NotI | from Addgene | Fluorescent protein | |
| | | #132706 | | |
| Mito-BFP | XhoI-MTS-BFP | Purchased | Mitochondrial | Used in PMID: |
| | NotI | from | matrix. Fluorescent | 27184847 |
| | | Addgene# | protein | |
| | | 49151 | | |
| CD63-V5-APEX2 | KpnI-CD63- | CMV-F/pCDNA5 | Late endosome/ | |
| | BamHI-V5 | | lysosomal membrane | |
| | APEX2-NotI | | V5: | |
| | | | GKPIPNPLLGLDST | |
| | | | (SEQ ID NO: 1) | |
| TOM20-V5- APEX2 | KpnI-TOM20- | CMV-F/pCDNA5 | Outer-mitochondrial | Used in |
| | linker-NheI-V5 | | membrane V5: | Lee SY et al. |
| | APEX2-NotI | | GKPIPNPLLGLDST | Cell Rep. |
| | | | (SEQ ID NO: 1) | 2016, 15. |
| | | | | 1837-1847 |
| | | | | (PMID: 27184847) |
| Matrix-V5-APEX2 | KpnI-MTS- | CMV-F/pCDNA5 | Mitochondrial matrix | Used in Lee SY |
| | BamHI-V5 | | V5: | et al. Cell Rep. |
| | APEX2-NotI | | GKPIPNPLLGLDST | 2016, 15. |
| | | | (SEQ ID NO: 1) | 1837-1847 |
| | | | | (PMID: 27184847) |

### 2-2. Cell Culture and Transfection

HEK293T cells were obtained from ATCC. HeLa cells were obtained from the Korean Cell Line Bank (KCLB No; 10002), which were used at <20 passages. HeLa cell lines were maintained in high-glucose Dulbecco's modified Eagle medium (DMEM) with 10% fetal bovine serum (FBS) at 37° C. in 5% CO₂(v/v). These cell lines were transiently transfected at 60-80% confluency using polyethyleneimine (PEI) and Lipofectamine2000 along with various plasmids.

### 2-3. Polymerization of JF Catalyzed by HRP

JF (400 μM) was prepared by dissolving the powder in DMSO. To induce the polymerization of JF, 10 μM of HRP was added along with 2 mM H₂O₂. JF-precipitate starts forming after 30 minutes of incubation with HRP and H₂O₂. This reaction was carried out at room temperature for 24 h to acquire the polymerized JF. The mixture was centrifuged at 15,000 g for 10 min to separate the polymerized JF product from non-reacted JF.

### 2-4. Live Cell APEX Labeling Using JF Probes

To visualize various APEX2-POIs in live cells, JF or JFA (50-100 μM) and 1 mM H₂O₂ were treated to APEX2-POI expressing cells during the live stage. After treatment, cells were washed thrice with DPBS and then fixed with 4% PFA in 1×DPBS for 15 minutes at room temperature. For immuno-fluorescence imaging, cells were permeabilized with −20° C. methanol for 5 minutes, followed by the standard immunofluorescence staining protocol. To detect JF or JFA fluorescence and immunofluorescence, we used the GFP channel (excitation at 488 nm), RFP channel (excitation at 561 nm), and Cy5 channel (excitation at 647 nm) of a confocal light microscope (FV3000, Olympus, Japan). HeLa cells were used for imaging experiments.

### 2-5. Fixed Cell APEX Labeling Using JF Probes.

HeLa cells were transiently transfected with the APEX2-POI construct using PEI when cells reached approximately 70% confluency, following the protocol provided by the production company of PEI. After transfection and protein expression, the cells were fixed with 4% PFA in 1×DPBS for 15 minutes at room temperature. Subsequently, the cells were washed three times with DPBS. For the APEX reaction, JFT probes (100-500 μM) and H₂O₂ (0.5-1 mM) were used in 0.1 M PHEMS buffer for the specified time points. The APEX2-mediated reaction was quenched using a quencher solution (a mixture of sodium ascorbate, trolox, and sodium azide, see PMID: 23371551), and the cells were washed three times with DPBS buffer. For immunofluorescence imaging, the cells were permeabilized with −20° C. methanol for 5 minutes, followed by the standard immunofluorescence staining protocol. The detection of JFT fluorescence and immunofluorescence was performed using the GFP channel (excitation at 488 nm), RFP channel (excitation at 561 nm), and Cy5 channel (excitation at 647 nm) of a confocal light microscope 428 (FV3000, Olympus, Japan).

### 2-6. Immunoblotting and Immunofluorescence

For the western blot analysis of JFA-labeled proteins, HEK293T cells were transiently transfected by TOM20-V5-APEX2 construct with PEI followed by the protocol of the production company of PEI when cells were around 70% confluent. JFA (50 M) and 1 mM H₂O₂ were treated for 1 min and cells were washed 3 times using DPBS. After the washing step, cells were lysed using RIPA buffer including 1× protease inhibitor cocktail for 10 min at room temperature. After transferring these lysates to e-tube, samples were vortexed for 2-3 min at room temperature. Using centrifugation at 5000×g for 10 min at 4° C., the pellet and supernatant fractions were separately obtained and copper-click reaction were conducted with. 50 μM biotin-azide, 100 μM THPTA, 100 M CuSO₄ and 2.5 mM sodium ascorbate for 15 mins. After click reaction, further lysis of pellet was done using 1 M Tris (pH 8.8). The supernatant and pellet samples were boiled with 1×SDS-PAGE loading buffer at 95° C. for 5 min. After resolving protein samples using SDS-PAGE and transferring protein samples to the nitrocellulose membrane, immunoblotting analysis was performed using antibodies. Membranes were blocked for 30 min with 2% skim milk solution at room temperature. After washing three times with TBST, at room temperature, primary antibodies, namely anti-V5 (Invitrogen, cat. No. R960-25, 1:10,000 dilution) was incubated for 1 h at room temperature. After washing three times with TBST, secondary antibodies, anti-mouse-HRP (Bio-rad, cat. No. 1706516, 1:3,000 dilution) were incubated for 30 min at room temperature. After washing three times with TBST at room temperature, results of immunoblotting assay were obtained using ECL solution using Ctyiva AI 600.

For immunofluorescence imaging, fixed and permeabilized cells (see “Fixed Cell APEX labeling using JF probes”) was incubated in blocking solution with appropriately diluted antibodies (anti-V5) (Invitrogen, cat. No. R960-25, 1:5000 dilution), and Alexa Fluor-labeled secondary antibodies (mouse anti-Alexa Fluor 647) (Invitrogen, cat. No. S21374, 1:1000 dilution) with extensive washes. Immunofluorescence images were obtained and analyzed on an Olympus Fluoview FV3000 (Seoul National University, Seoul, Republic of Korea) with an objective lens (UPLXAPO 100×/1.40 OIL) in GFP (488 nm laser), RFP (561 nm laser) and Cy5 (647 nm laser) channels, which was controlled with Olympus software.

2-7. CLEM Imaging of JFT1-Labeled Region with TEM (Post-Embedding Osmium Treatment Procedure)

For CLEM imaging of JFT1-labeled cells via post-embedding osmium treatment procedure (FIG. 2C, upper), cells were cultured in 35-mm glass grid-bottomed culture dishes (MatTek life sciences, MA, USA) to 30-40% confluency (5×10⁴ cells). Then, cells were transfected with APEX2-POI plasmids using Lipofectamine 2000 (Life Technologies, Carlsbad, CA, USA). Next day, cells were fixed with 2.5% glutaraldehyde (Electron Microscopy Sciences, cat. No. 16200) and 2% paraformaldehyde (Electron Microscopy Sciences, cat. No. 19210) in 0.1 M cacodylate solution (pH 7.0) for 30 min at 4° C. After washing, 20 mM glycine solution was used for quenching unreacted aldehyde. Next, JFT1 probe labeling with 100-300 μM JFT1 with 0.5-1 mM H₂O₂ was conducted for 5 min. For registering the JFT1 fluorescence, cells were imaged with a confocal light microscope (A1 Rsi/Ti-E, Nikon, Japan) using GFP (excitation at 488 nm) and RFP channel (excitation at 561 nm). Next, the JFT1-labeled cells were post-fixed with 2% osmium tetroxide (OsO₄) containing 1.5% potassium ferrocyanide in distilled water for 30 min at 4° C. and en bloc in 1% uranyl acetate (EMS, USA, cat. No. 22400) overnight and dehydrated with a graded ethanol series. The samples were then embedded with an EMBed-812 embedding kit (Electron Microscopy Sciences, USA, cat. No. 14120) and polymerized in oven at 60° C. The polymerized samples were sectioned (60-nm section) with an ultramicrotome (UC7: Leica Microsystems, Germany), and the sections were mounted on copper slot grids with a specimen support film. Sections were stained with UranyLess (Electron Microscopy Sciences, cat. No. 22409) and lead citrate (Electron Microscopy Sciences, cat. No. 22410) then viewed on a Tecnai G2 transmission electron microscope (TEM, ThermoFisher, USA). Confocal micrographs were produced as high-quality enlarged images using PhotoZoom Pro 8 software (Benvista Ltd., Houston, TX, USA). To register the enlarged fluorescence images to the electron micrographs we used the ImageJ BigWarp software package.

For the incubation step of JFT1, the condition of 100 μM JFT1 for 5 min in non-embedded sample imaging was initially optimized. Since CLEM requires long and intensive sample preparation steps that can affect the fluorophore in the sample, the concentration of JFT1 to retain its fluorescence was increased. We optimized the CLEM condition for pre-embedding to 500 M JFT1 for 5 min and the condition for post-embedding was optimized to 300 μM JFT1 for 5 min.

2-8. CLEM Imaging of JFT1-Labeled Region with SEM (Pre-Embedding Osmium Treatment Procedure)

To obtain the SEM-based CLEM images via pre-embedding osmium treatment procedure (FIG. 2C, lower), cells were fixed with 2.5% glutaraldehyde (Electron Microscopy Sciences, cat. No. 16200) and 2% paraformaldehyde (Electron Microscopy Sciences, cat. No. 19210) in 0.1 M cacodylate solution (pH 7.0) for 30 min at 4° C. After washing, 20 mM glycine solution was used for quenching unreacted aldehyde. Next, JFT1 probe labeling with 500 μM JFT1 and 0.5-1 mM H₂O₂, was conducted for 5 min. Next, JFT1-labeled cells were post-fixed with 0.4% osmium tetroxide (OsO₄) containing 1.5% potassium ferrocyanide in distilled water for 30 min at 4° C. and en bloc in 1% uranyl acetate (EMS, USA, cat. No. 22400) overnight and dehydrated with a graded ethanol series. The samples were then embedded with an EMBed-812 embedding kit (Electron Microscopy Sciences, USA, cat. No. 14120) and polymerized in an oven at 60° C. Resin embedded samples were sectioned (100-nm sections) from the block using an ultramicrotome (UC7: Leica Microsystems, Germany) with a diamond knife (AT-4 Diamond knife, Leica Microsystems, Germany). The indium-tin-oxide (ITO)-coated glass coverslips were cleaned manually with isopropanol and the sections were mounted onto ITO-coated coverslips. To obtain the fluorescence image, sections were observed with a confocal light microscope (A1 Rsi/Ti-E, Nikon, Japan) using RFP channel (561 nm laser). Ultrathin sections on ITO-coated coverslips were then stained with UranyLess for 2 min and 3% lead citrate for 1 min, and then imaged by backscatter detector on a Gemini 300 SEM (ZEISS, Germany) at an accelerating voltage of 5 kV. Image resolution in the x-y plane was 5 nm/pixel. The correlation between the fluorescent images and the electron microscopy images were performed by Adobe Photoshop.

Fiji/ImageJ was used for calculating Pearson coefficient in colocalization results and measurement of lysosome diameter and area in the boundary of each cell. All data fitting and statistical analysis were performed using GraphPad Prism 7/9.1.2 software. Exact n values, and statistical significance are indicated in each figure and figure legend. Pearson correlation was conducted using image J software program (NIH).

2-9. Quantification of Lysosome and Mitochondria Contact from Fluorescent Images as Well as Lysosome Diameter.

Fluorescently labeled lysosome (JFT1-labeled or Lysotracker Red, RFP channel) and mitochondria (mito-mEosEM, GFP channel) area were analyzed using Fiji/ImageJ (National Institutes of Health, USA). Area corresponding to the colocalization of lysosome-mitochondria were delimited manually by thresholding keeping the same threshold for control. The colocalized area was measured for 5 cells in each group. To measure lysosome diameter, we utilized Fiji/ImageJ. Initially, the images were converted to 8-bit and the pixel measurements were calibrated using a scale bar. Circular lysosomes were then selected and a line representing the diameter of each selected lysosome was drawn, referencing the limiting membrane according to the JFT1 fluorescence signal. Subsequently, we utilized the “analyze and measure” feature of ImageJ, which generates a table with various parameters such as area and length. To convert the length from pixels to micrometers, we applied the following formula: (pixel diameter/pixels on the scale bar)×10 μm. Once the values were converted, they were plotted and statistical analysis for unpaired t-test was performed using Prism GraphPad software (Scerra et al., 2021).

### 2-10. Treatment of Bafilomycin and U18666A

HeLa cells transiently expressing TMEM192-V5-APEX2 were plated on coverslips (thickness no. 1.5, radius 18 mm). The cells were then treated with DMSO or U18666A (2 ug/ml), Bafilomycin A1 (100 nM) in DMEM for 18 h at 37° C. and washed three times with cold DPBS. The cells were fixed in 4% paraformaldehyde for 15 min at room temperature. Next, JFT1 labeling was done using 100 μM of the probe and 1 mM H₂O₂ for 5 min. Reaction was quenched and cells were permeabilized for immunofluorescence imaging with cold methanol for 5 min at −20° C., followed by washing with DPBS and blocking for 1 h with 2% bovine serum albumin in DPBS at room temperature.

### 2-11. Product Information

### Example 2

| | | |
|---|---|---|
| REAGENT or SOURCES | SOURCE | IDENTIFIER |

Antibodies

| | | |
|---|---|---|
| Anti-V5 Tag Monoclonal | Invitrogen | Cat # R960-25; |
| Antibody (mouse) | | RRID: AB_2556564 |
| Alexa Fluor 647 IgG | Invitrogen | Cat # A28181; |
| mouse | | RRID: AB_2536165 |
| Streptavidin-HRP | Thermo Fisher Scientific | Cat # 21126 |
| Streptavidin, Alexa Fluor | Invitrogen | Cat # S21374 |
| 647 conjugate | | |

Chemicals, Peptides, and Recombinant Proteins

| | | |
|---|---|---|
| Desthiobiotin Phenol | Mishra et.al | PMID: 36265183 |
| RIPA lysis buffer | ELPISBIO | Cat # EBA-1149 |
| Protease inhibitor cocktail | Invitrogen | Cat # 78438 |
| PEI (polyetherimide) | Polyscience | Cat # 23966-1; |
| | | CAS: 9002-98-6 |
| 4% paraformaldehyde solution | Chembio | Cat # CBPF-9004 |
| 5X SDS loading buffer | Biosesang | Cat # SF2002-110-00 |
| Glutaraldehyde | Electron Microscopy | Cat #16200 |
| | Sciences | |
| Osmium tetroxide | Electron Microscopy | Cat #19150 |
| | Sciences | |
| Uranyl acetate | Electron Microscopy | Cat #22400 |
| | Sciences | |
| EMBed-812 embedding kit | Electron Microscopy | Cat #14120 |
| | Sciences | |
| Uranyless | Electron Microscopy | Cat #22409 |
| | Sciences | |
| Lead citrate | Electron Microscopy | Cat#22410 |
| | Sciences | |

Deposited data
Experimental models: Cell lines

| | | |
|---|---|---|
| Human: HEK293T | ATCC | CRL-3216 |
| Human: HeLa | Dr. Mun at Korea Brain | N/A |
| | Research Institute | |

Software and Algorithms

| | | |
|---|---|---|
| Fiji/Image J | NIH | https://imagej.nih.gov/ij/ |
| Adobe Photoshop | | |
| GraphPad Prism 7/9.1.2 | GraphPad | https://www.graphpad.com/ |

### Example 3

### Effect of JF Probe in Live and Fixed APEX-Expressing Cells

To explore the potential of JF as a fluorescence amplification substrate for APEX, JF and its structural variants, alkyne-Jenfluor (JFA) and Jenfluor triazole (JFT) were synthesized. JFA features a copper-click enrichable alkyne moiety PEGs (Yi et al., 2019, Dyes and Pigments 166, 460-466), while JFT probes (JFT-PEG1, JFT-PEG2, and JFT-PEG3) were synthesized via the copper-click reaction with azido-PEGs.

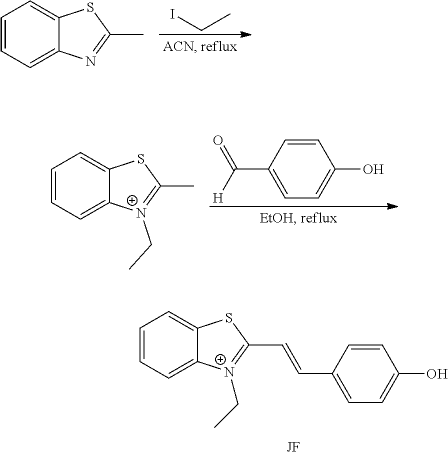

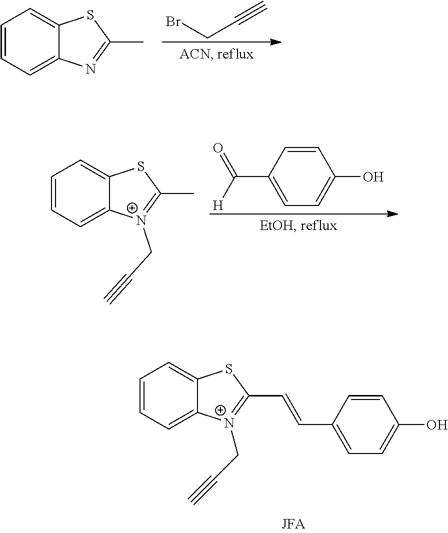

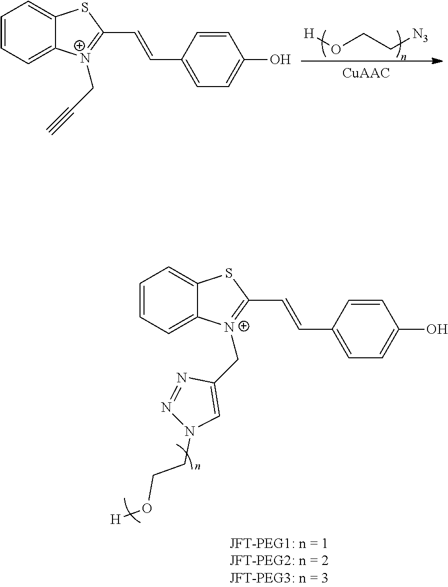

JF probes displayed light absorption around 410 and 520 nm and exhibited red fluorescence (λmax: 575 nm, excitation at 520 nm) in neutral pH buffer. These photophysical properties render the JF probes suitable for detection in the RFP channel (excitation at 561 nm, fluorescence emission filter: 565-620 nm). In a free-solution condition, JFT1 showed green fluorescence in an acidic buffer (pH<7). The absorbance spectra largely shifted from 515 (pH 7.4) to 416 nm when the probe was incubated at pH 6.0. Moreover, JFT1's green fluorescence intensity at Imax of 520 nm was significantly increased at an acidic pH (pH<7, excitation wavelength: 405 or 450 nm). This property enables the detection of JF probes in the GFP channel; however, the fluorescence intensity was negligible compared to that of GFP or GFP variant proteins (mEosEM etc.).

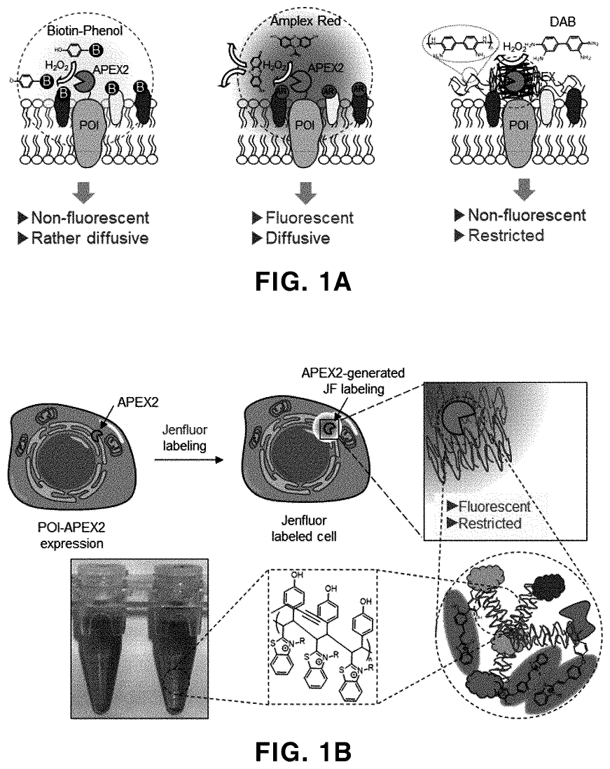

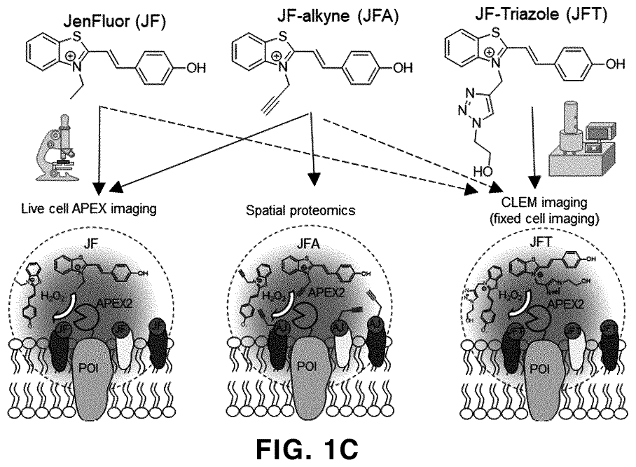

Due to the presence of a phenol group, it was expected that APEX2 would convert the phenol group of JF probes to a phenoxy radical state in the presence of H₂O₂. These radicals would then react with local proteins or with each other to form a locally precipitated polymer, ultimately generating a spatially restricted and amplified fluorescent signal by peroxidase. The present inventor confirmed that JF exhibited macroscopic precipitate deposition upon the HRP-mediated reaction, indicative of its potential for polymerization by peroxidase (FIG. 1B). These APEX-activable JF probes enabled fluorescent visualization of the localization of various APEX2-conjugated POIs and enabled CLEM sampling under the fixed conditions at a reasonably high resolution (FIG. 1C).

Further, the APEX reaction in live cells utilizing the JF probes in cells expressing TOM20-V5-APEX2 at the cytosolic face of the outer mitochondrial membrane (OMM) were examined. Each JF probe (JF, JFA, JFT1, JFT2, and JFT3) was pre-incubated with the cells for 5 min, followed by the addition of 1 mM H₂O₂ and incubation for 1 min to activate the peroxidase. JF and JFA showed red fluorescence stains in TOM20-V5-APEX2-expressing cells, whereas the PEG probes did not show any fluorescence in live cells owing to their membrane impermeability. In the JFA-labeled sample, it was observed the selective biotin conjugation at the alkyne moieties of JFA-labeled proteins using a fixed-cell copper-click reaction with biotin-azide, although the pattern was found to be diffusive. Western blot analysis demonstrated highly cross-linked JFA-labeled proteins in the pellet fraction. This result support the formation of JFA-containing polymers at the labeled proteins (FIG. 1B). Additionally, in untransfected cells or cells not treated with H₂O₂, JFA exhibited self-localization to the vesicles or other intracellular compartments, which was possibly attributed to the nitrogen group.

Next, a fixed-cell APEX reaction using the same construct was conducted and all JF probes, including JFT1-3 were tested for their reactivity with TOM20-V5-APEX2. Line scan analysis of the JF probe-labeled region (RFP signal) alongside observation of the enzyme localization pattern (TOM20-V5-APEX2 stained with anti-V5) demonstrated that JFT1 exhibited the most optimal and restricted labeling pattern among all the tested JF probes (Data not shown). The fluorescence signal and diffusiveness of JFT1 labeling were enhanced by increasing the probe incubation time or probe concentrations. To achieve strong fluorescence intensity and a restricted staining pattern under optimized JFT1 staining conditions, an incubation time of 5 minutes and a concentration of 100 μM were selected.

It was also compared the restriction of fluorescence labeling by JFT1, Amplex Red, and desthiobiotin-phenol (DBP) using TOM20-V5-APEX2 in a fixed sample. JFT1 exhibited the highest Pearson correlation coefficient (r=0.91; FIG. 2A) between the probe-labeling region (RFP signal) and the enzyme-expressed region (anti-V5 stain). Moreover, line scan analysis showed that JFT1 labeling had the most well-matched pattern with the anti-V5 stain compared to the other probes (FIG. 2B).

### Example 4

### Suitability of CLEM Imaging for Ultrastructure of Cellular Organelles

Two CLEM approaches were tested: pre-embedding OsO₄ treatment and post-embedding OsO₄ treatment (FIG. 2C). The pre-embedding OsO₄ treatment typically exhibits a strong correlation between fluorescence and electron microscopy images. However, this method may lead to fluorescence signal quenching due to the osmium treatment or high-temperature resin-embedding procedures.

Given the significance of osmium staining in EM sample preparation, the preservation of JF and JFT1 fluorescence after OsO₄ treatment was tested. Remarkably, both JF- and JFT1-stained APEX2-expressing regions retained 70-80% of the original osmium-resistant fluorescence intensity even after osmium treatment (0.1% OsO₄), facilitating their detection using confocal microscopy (FIG. 2D). To further evaluate the performance of JFT1 under the harsh pre-embedding condition, JFT1-stained (500 μM JFT1, 1 mM H₂O₂, 5 min labeling) fixed Matrix-V5-APEX2 cells were generated and processed through pre-embedding steps, including 0.4% OsO₄ incubation, dehydration, resin embedding, and sectioning procedures. Notably, the fluorescence signal of JFT1 was well-preserved in the 100 nm ultrathin section, and the JFT1-labeled region precisely matched the mitochondrial localization (FIG. 2E). This result demonstrates that JFT1 can effectively serve as a fluorescent probe for APEX-CLEM imaging.

### Example 5

### Fluorescence Signal Amplification Via APEX (FLEX) of JF Probes in Cell Organelles

It was assessed whether JFT1 could react with APEX2 targeting various organelles under fixed conditions. As shown in FIG. 3, JFT1 exhibits reasonable fluorescence staining for various organelle-targeted APEX2. In all the tested APEX2-POIs, JFT1 staining (RFP channel) accurately overlapped with enzyme localization (anti-V5 stain, Cy5 channel). Notably, as observed on the cytosolic side of the OMM (TOM20-V5-APEX2), JFT1 generated a highly restricted fluorescence signal in all non-membrane locations, including the cytosolic side of the ER membrane (APEX2-V5-SEC61B), cytosolic membrane of lipid droplets (APEX2-V5-PLIN2), cytosolic side of the endolysosomal membrane (CD63-V5-APEX2), and cytosolic side of the lysosomal membrane (TMEM192-V5-APEX2). Furthermore, JFT1 effectively generated a fluorescence signal with the APEX2-tagged POIs (APEX2-POIs) within specific compartments, including the mitochondrial matrix (Matrix-V5-APEX2), nucleus (V5-APEX2-NLS), and ER lumen (SEC61B-V5-APEX2) under fixed-cell conditions. It was confirmed that the JFT1-labeling patterns of V5-APEX2-NLS (nucleus), Matrix-V5-APEX2 (mitochondrial matrix), and APEX2-V5-PLIN2 (lipid droplets) were well matched to the patterns of the subcellular fluorescent markers DAPI (nucleus), Mito-BFP (mitochondria), and LipiDye (lipid droplets), respectively. These findings validate the wide applicability of JFT1 for APEX2-mediated fluorescence signal amplification (FLEX) in various subcellular organelles. For this experiment, it was utilized PHEMS buffer (100 mM, pH 7.4), which is commonly employed for CLEM sample preparation. Importantly, JFT1 probe exhibited a strong red fluorescence signal (RFP channel) and a weak green fluorescence signal (GFP channel. The weak green fluorescence suggests that JFT1 may have a partially protonated structure when labeled on the protein or polymerized by APEX2, even when the reaction is conducted under a slightly basic condition (i.e., PHEMS buffer, pH 7.4).

### Example 6

### CLEM Imaging of Various Cellular Organelles Targeting APEX2 Using JFT1

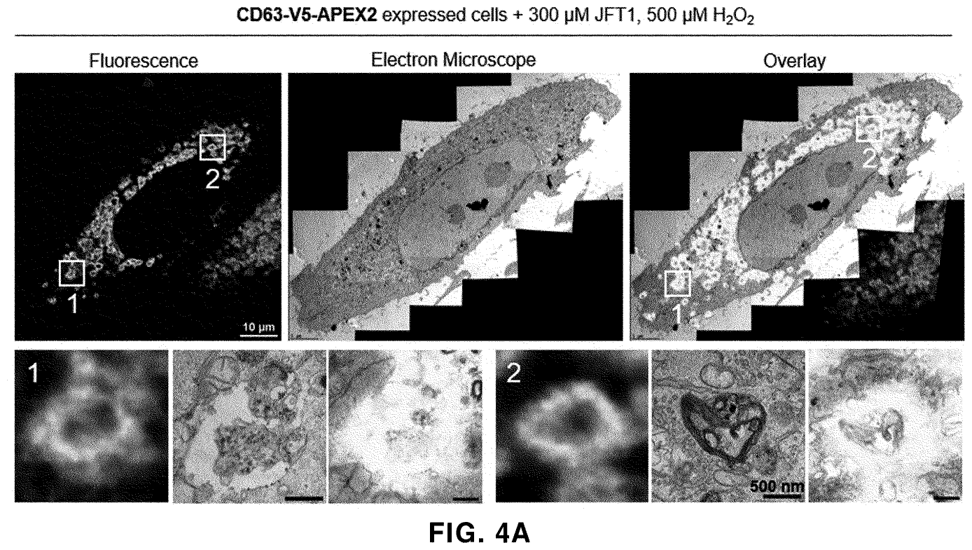

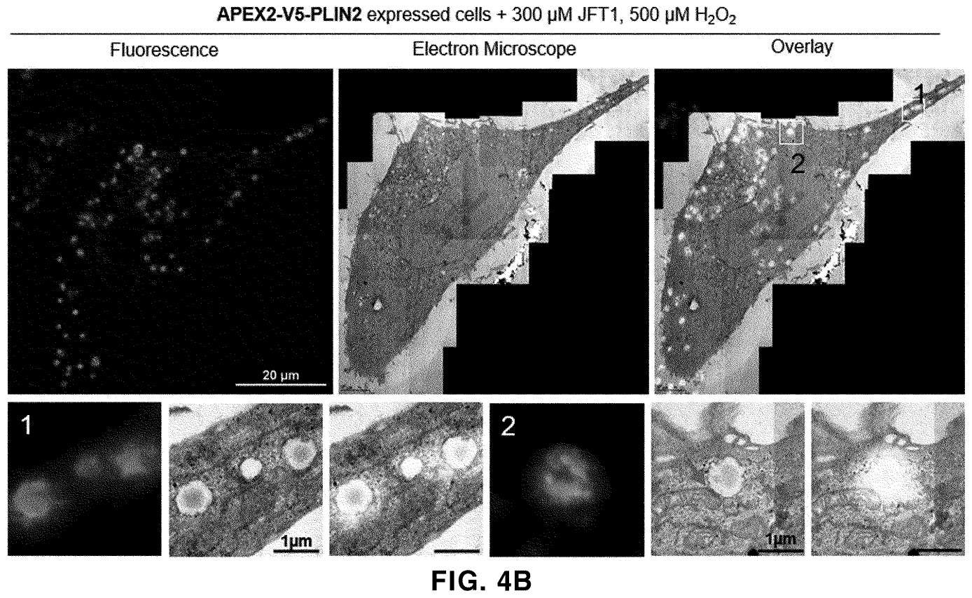

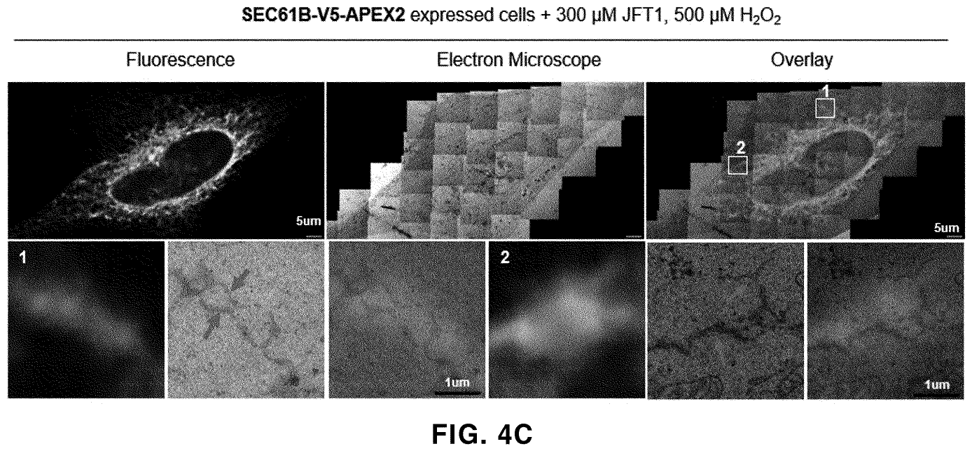

After confirming the APEX-labeling activity of JFT1 in fixed cells, it was attempted to obtain CLEM images of various organelle-targeted structures by following the post-embedding OsO₄ treatment procedure. For this experiment, CD63-V5-APEX2 construct, which is expressed in the endolysosome membrane and multivesicular body (MVB), was used (FIG. 4A). CD63-V5-APEX2 transfected cells were fixed, and then treated with JFT1 (300 μM) and H₂O₂ (500 μM) for 5 min. After labeling, the JFT1 fluorescence signal from CD63-V5-APEX2 was detected using confocal microscopy (FIG. 4A). Subsequently, JFT1-labeled cells were treated with 2% OsO₄ and dehydrated using graded ethanol. Following the Epon-embedding process, 60-nm ultrathin sections were generated using a diamond knife and visualized under a transmission electron microscope (TEM). By employing this sequential imaging procedure, it was overlapped the confocal microscope images containing JFT1-labeled regions with the TEM images. This approach clearly visualized the ultrastructure of the MVBs where CD63-V5-APEX2 was localized using the fluorescence signals of JFT1 (FIG. 4A).

Similarly, it was obtained high-quality CLEM images for APEX2-V5-PLIN2 (FIG. 4B) and SEC61B-V5-APEX2 using JFT1 (FIG. 4C). These imaging results provided clear visualization of the respective localization of these APEX2-POIs at the lipid droplet (LD) membrane or in the ER luminal space, with the combined resolution of confocal and electron microscopes. Labeling LDs with JFT1 using APEX2-V5-PLIN2 showed distinctive red fluorescence signals localized within the LD, distinguishing it from the adjacent regions (FIG. 4B). Notably, organelle contacts between LDs and various subcellular organelles (e.g., LD-ER, LD-nucleus, LD-mitochondria, marked with red arrows in FIG. 4B) were well-preserved in our CLEM images using JFT1 and APEX2-V5-PLIN2. This result implies that LD communicates with other cellular compartments.

### Example 7

### FLEX-CLEM Imaging of Organelle Structure and Lysosome Interactions

Next, interactions between lysosomes and mitochondria were investigated using the FLEX approach. Lysosomes are highly dynamic structures involved in various physiological and pathophysiological processes in response to diverse stimuli. It was cloned TMEM192-V5-APEX2, expressing APEX2 in the C-terminal domain of the cytosol-facing region of the TMEM192 protein (Behnke et al., (2011) Biochem J 434, 219-231; Schroder et al., (2010). Biol Chem 391, 695-704). Through confocal and EM imaging analyses, followed by post-embedding OsO₄ treatment, it was observed well-restricted lysosomal patterns generated by JFT1 in HeLa cells expressing TMEM192-V5-APEX2 (FIG. 5A).

Considering the dynamic nature of lysosomes, which adjust their size, number, and position in response to internal or external stressors, it was further explored whether FLEX could characterize dynamically remodeled lysosomal structures in response to external stress. First, it was tested the effects of Bafilomycin A1 (BFA, 100 nM), a well-known inhibitor of lysosomal V-ATPase, and found that BFA-treated cells exhibited an increased lysosomal diameter compared to that of the control sample (i.e., DMSO treatment) (FIG. 5A-B). The lysosomes exhibited a mean (±SEM) diameter of 0.43 μm (±0.02) during DMSO treatment, which increased by 2.7-fold to 1.20 μm (±0.04) under BAF treatment.

As BFA treatment is known to enhance mitochondria-lysosome interactions, mitochondria-targeted mEosEMs (mito-mEosEMs) and TMEM192-V5-APEX2 were co-expressed in HeLa cells. In these cells, we observed similar lysosomal morphological changes using JFT1 labeling by TMEM192-V5-APEX2, along with more merged patterns between mitochondria and lysosomes (data not shown).

Importantly, it was confirmed that the green fluorescence signal of JFT1 was negligible compared to that of mito-mEosEM under the same laser and detector settings, and mito-mEosEM was predominantly detected in the GFP channel. In the RFP channel, the red fluorescence signal of JFT1 was distinct and did not overlap with that of mito-mEosEM at all. This result demonstrates that two-color CLEM imaging is enabled by mEosEM and FLEX with JFT1.

### Example 8

### CLEM Imaging of TMEM192-V5-APEX2 and Mito-mEosEM Under BFA Treatment Conditions Using JFT1

Using JFT1, CLEM imaging of TMEM192-V5-APEX2 and mito-mEosEM was performed under BFA-treated conditions (FIG. 5C). Interestingly, in the BFA-treated sample, it was observed accumulation of TMEM192-positive lysosomal vesicles with damaged mitochondria (marked with red arrows, FIG. 5C). This accumulation may be attributed to the inhibition of lysosomal acidification by BFA, suggesting an impact of lysosomal perturbation on mitochondrial physiology.

Next, U18666A (2 μg/ml, 18 h), another lysosome perturbation molecule known to accumulate cholesterol in the lysosome by inhibiting the cholesterol transport protein, NPC1, was tested (Hoglinger et al., (2019) Nature Communications 10, 4276). Similar to BFA-treated cells, the cells treated with U18666A also displayed significant enlargement of the lysosomes. The mean (±SEM) lysosomal diameter was 0.39 μm (±0.01) during DMSO treatment, which expanded by 2.2-fold to 0.87 μm (±0.04) following U18666A treatment. Additionally, this treatment led to enhanced co-localization between mitochondria and lysosomes.

Using mito-mEosEM and TMEM192-V5-APEX2 co-expressed HeLa cells, CLEM imaging was performed under U18666A treatment, revealing increased interactions between mitochondria and lysosomes, consistent with previous studies. In the EM imaging results of the same sample, it was observed that mitochondria-lysosome contacts under U18666A treatment differed from those in the BFA-treated sample. Specifically, the mitochondria appeared to be in contact with or partially engulfed by TMEM192-positive lysosomes (FIG. 5D), resembling the recently reported self-mitophagy process (Hao et al., (2023) Nature Communications 14, 4105).

Since these differences cannot be discerned with optical microscope imaging resolution, the present invention, two-color CLEM imaging provides a valuable approach to study dynamic organelle contact networks at high resolution.

While the specific parts of the present invention have been described in detail above, it will be apparent to those skilled in the art that such specific descriptions are merely preferred embodiments and that the scope of the present invention is not limited thereby. Accordingly, the actual scope of the present invention will be defined by the appended claims and their equivalents.

### National Research and Development Project Supporting this Invention

[Project Unique Number] 1711155246

[Project Number] 2022R1A2B5B03001658

[Ministry] Ministry of Science and ICT

[Project Management (Specialized) Institution] National Research Foundation of Korea

[Research Program] Basic Research Program (Ministry of Science and ICT)

[Research Project Title] Identification of a New Retrograde Signaling Pathway in Mitochondria

[Project Performing Institution] Seoul National University

[Research Period] 2022.03.01˜2025.02.28

[National Research and Development Project Supporting This Invention]

[Project Unique Number] 1465039656

[Project Number] HU23C020400

[Ministry] Ministry of Health and Welfare

[Project Management (Specialized) Institution] Korea Health Industry Development Institute

[Research Program] Dementia Research and Development Program (Ministry of Health and Welfare)

[Research Project Title] Identifying Risk Factors for Dementia Using a Brain Spatial Molecular Mapping Platform

[Project Performing Institution] Seoul National University

[Research Period] 2023.04.01˜2025.12.31

1. A compound of the following Chemical Formula I or Chemical Formula II: 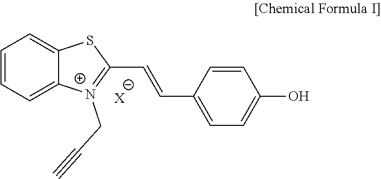
(X represents an atom or a molecular group that acts as a monovalent anion); 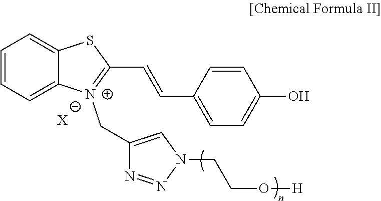
(n represents 1, 2, 3, 4, or 5, and X represents an atom or a molecular group that acts as a monovalent anion).

2. A method for preparing the compound of Chemical Formula I of claim 1, comprising:
(a) synthesizing (E)-2-(4-hydroxystyryl)-3-(prop-2-yn-1-yl)benzo[d]thiazol-3-ium through a Knoevenagel condensation reaction between 4-hydroxybenzaldehyde and 2-propargyl benzothiazolium salt.

3. A method for preparing the compound of Chemical Formula II of claim 1, comprising:
(a) synthesizing (E)-2-(4-hydroxystyryl)-3-(prop-2-yn-1-yl)benzo[d]thiazol-3-ium through a Knoevenagel condensation reaction between 4-hydroxybenzaldehyde and 2-propargyl benzothiazolium salt; and
(b) synthesizing the compound of Chemical Formula II by reacting (E)-2-(4-hydroxystyryl)-3-(prop-2-yn-1-yl)benzo[d]thiazol-3-ium with Azido-PEGn (where n is an integer from 1 to 5) through a Copper-Catalyzed Azide-Alkyne Cycloaddition (CuAAC) reaction.

4. The method of claim 2, wherein step (a) comprises adding a pyridine catalyst and stirring at a temperature of 50-60° C. for 8-16 hours to carry out the Knoevenagel condensation reaction.

5. The method of claim 3, wherein step (b) comprises adding NaHCO₃, sodium ascorbate, and CuSO₄·5H₂O, and stirring at room temperature for 20 minutes to 3 hours to carry out the Copper-Catalyzed Azide-Alkyne Cycloaddition (CuAAC) reaction.

6. The method of claim 2, wherein step (a) further comprises precipitation, filtration, washing, and/or separation after the reaction is completed to obtain the synthesized (E)-2-(4-hydroxystyryl)-3-(prop-2-yn-1-yl)benzo[d]thiazol-3-ium.

7. The method of claim 3, wherein step (b) further comprises extraction, drying, and/or purification after the reaction is completed to obtain the compound of Chemical Formula I.

8. A fluorescent probe comprising the compound of claim 1.

9. The fluorescent probe of claim 8, wherein the compound generates a fluorescence signal amplified by a peroxidase.

10. The fluorescent probe of claim 9, wherein the compound amplifies a localized fluorescence signal within a cell in vitro.

11. The fluorescent probe of claim 9, wherein the peroxidase is selected from the group consisting of APEX, APEX2, and HRP.

12. The fluorescent probe of claim 8, wherein the fluorescent probe is used for an electron microscopy or a correlative light and electron microscopy.

13. A method for intracellular peroxidase-mediated fluorescence signal amplification, comprising incubating the fluorescent probe of claim 8 with hydrogen peroxide in a cell overexpressing a peroxidase.

14. The method of claim 13, wherein the fluorescent probe is applied at a concentration of 10-1000 μM, hydrogen peroxide (H₂O₂) is applied at a concentration of 0.1-5 mM, and the incubation is carried out for 1-10 minutes.

15. The method of claim 13, wherein the compound in the fluorescent probe generates a fluorescence signal amplified by a peroxidase.

16. The method of claim 13, wherein the compound in the fluorescent probe amplifies a localized fluorescence signal within a cell in vitro.

17. The method of claim 13, wherein the peroxidase is selected from the group consisting of APEX, APEX2, and HRP.

18. The method of claim 13, wherein the fluorescent probe is used for an electron microscopy or a correlative light and electron microscopy.

19. A method for identifying an intracellular location of a protein of interest (POI), comprising:
(a) expressing a peroxidase fused with a protein of interest in a cell;
(b) incubating the cell after treatment with the fluorescent probe of claim 8 and hydrogen peroxide; and
(c) identifying a cellular organelle where amplified fluorescence signals from the fluorescent probe are observed.
